(19) United States
(12) Patent Application Publication
(10) Pub. No.: US 2018/0332878 A1
(43) Pub. Date: Nov. 22, 2018

(54) BITTER TASTE INHIBITOR

(71) Applicant: NAGAOKA CO., LTD., Osaka (JP)

(72) Inventors: Seiji HASHIMOTO, Osaka (JP); Kimihiro HIGASHI, Tokyo (JP); Tatsunori TAKIMOTO, Tokyo (JP); Jyunji TOBARI, Tokyo (JP); Nozomu HIRATA, Osaka (JP)

(73) Assignee: NAGAOKA CO., LTD., Osaka (JP)

(21) Appl. No.: 15/983,762

(22) Filed: May 18, 2018

(30) Foreign Application Priority Data
May 18, 2017 (JP) 2017-098703
Nov. 8, 2017 (JP) 2017-215706

(51) Int. Cl. A23L 27/20; A23L 27/00; C12G 3/06; A23L 2/56; A23F 5/46; A23F 3/40; A23G 1/32

(52) U.S. Cl. CPC A23L 27/2024; A23L 27/86; C12G 3/06; A23V 2002/00; A23F 5/465; A23F 3/405; A23G 1/32; A23L 2/56

(57) ABSTRACT

A bitter taste inhibitor according to the present disclosure contains isobutyl angelate as an active and a given solvent or a given excipient.

### BACKGROUND OF THE INVENTION

### 1. Technical Field

The present disclosure relates to a bitter taste inhibitor, a food or beverage having a reduced bitter taste, and a method for inhibiting a bitter taste in a food or beverage.

### 2. Background

The basic tastes experienced upon the intake of foods, beverages and the like include a sweet taste, a sour taste, a salty taste, a bitter taste and an umami taste. The flavor of a food or beverage is brought about by a combination of these five tastes.

For example, when the combination of these tastes in a food or beverage is imbalanced, a particular taste stands out and therefore a sense of discomfort is left upon the ingestion of the food or beverage. Particularly when a bitter taste spreads in the mouth, many persons feel unpleasant. Then, methods for inhibiting or masking a bitter taste have been reported (see, for example, Japanese Unexamined Patent Publication No. 2015-19655, Japanese Unexamined Patent Publication No. 2011-15632, Japanese Unexamined Patent Publication No. 2012-121869 and Japanese Unexamined Patent Publication No. 2012-121868)

In Japanese Unexamined Patent Publication No. 2015-19655, it is described that a yeast extract containing a peptide, RNA and a free amino acid is used for the inhibition of a bitter taste. In Japanese Unexamined Patent Publication No. 2011-15632, it is described that a mammal-derived collagen peptide is used for the inhibition of a bitter taste. In Japanese Unexamined Patent Publication No. 2012-121869, an inhibitor for a catechin-derived bitter taste is disclosed, which contains, as an active ingredient, at least one component selected from Myristica fragrans, Scopolia acutangula, Euphorbia lathyris LINN., Solanum lyratum, Sambucus williamsii, Eleutherococcus senticosus, Sesamum indicum, Morus alba, Caesalpinia sappan L., Monascus purpureus, Perilla frutescens var. crispa, Allium tuberosum Rottler ex Spreng, Ocimum basilicum, Draba nemorosa, Impatiens balsamina and extracts thereof. In Japanese Unexamined Patent Publication No. 2012-121868, an inhibitor for a catechin-derived bitter taste is disclosed, which contains, as an active ingredient, at least one fragrance selected from methyl cinnamate, p-methoxy acetophenone, cinnamyl formate, cognac oil, cedrol, methyl anisate, perillaldehyde, tansy oil, γ-undecalactone, methyl benzoate, nerol, benzyl phenylacetate, rose oil, isopulegol, dodecanal, cumin oil and cyclohexadecanolide.

Peptides and amino acids, which are decomposition products of proteins, are known as taste components for umami taste and the like. Therefore, if a peptide or an amino acid is added to a food or beverage, the influence of the food or beverage may be altered or the influence of the food or beverage may be deteriorated even if the bitter taste of the food or beverage is reduced. The plant extracts disclosed in Japanese Unexamined Patent Publication No. 2012-121869 and the fragrances disclosed in Japanese Unexamined Patent Publication No. 2012-121868 may deteriorate the influence of foods or beverages and unpleasant odors may be imparted to the foods or beverages, even if a catechin-derived bitter taste in the foods or beverages is reduced.

### SUMMARY

A problem to be solved by the present disclosure is to provide a bitter taste inhibitor which can inhibit a bitter taste in a food or beverage without deteriorating the influence of the food or beverage itself.

The bitter taste inhibitor according to the present disclosure contains isobutyl angelate as an active and a solvent or an excipient. The solvent is at least one selected from the group consisting of ethanol, hydrous ethanol, glycerin, hydrous glycerin, propylene glycol, hydrous propylene glycol, vegetable oil, medium-chain triglyceride, triacetin. The excipient is at least one selected from the group consisting of dextrin and gum Arabic. The bitter taste inhibitor is effective to inhibit a bitter taste derived from at least one food stuff selected from the group consisting of citrus, coffee, tea, cacao and hop, or a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian.

The food or beverage having a reduced bitter taste according to the present disclosure contains: a food or beverage having a bitter taste derived from at least one foodstuff selected from the group consisting of citrus, coffee, tea, cacao and hop; and the above-mentioned bitter taste inhibitor, wherein the bitter taste inhibitor is contained in an amount of 0.05 to 2 ppm by mass in terms of an isobutyl angelate content. The food or beverage having a reduced bitter taste according to the present disclosure contains: a food or beverage having a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian; and the bitter taste inhibitor according to claim 6, wherein the bitter taste inhibitor is contained in an amount of 0.05 to 4 ppm by mass in terms of an isobutyl angelate content.

The method for inhibiting a bitter taste in a food or beverage according to the present disclosure includes adding 0.05 to 2 ppm by mass of isobutyl angelate to a food or beverage having a bitter taste derived from at least one foodstuff selected from the group consisting of citrus, coffee, tea, cacao and hop. The method for inhibiting a bitter taste in a food or beverage according to the present disclosure includes adding 0.05 to 4 ppm by mass of isobutyl angelate to a food or beverage having a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian.

According to the present disclosure, a bitter taste inhibitor can be provided, which can inhibit a bitter taste in a food or beverage without deteriorating the influence of the food or beverage itself. According to the present disclosure, a food or beverage having a reduced bitter taste can be provided, which is reduced in a bitter taste without deteriorating the influence of the food or beverage itself.

### DETAILED DESCRIPTION

The bitter taste inhibitor according to one embodiment of the present disclosure contains isobutyl angelate as an active ingredient. Isobutyl angelate is one of angelates, and has a structure represented by formula (I).

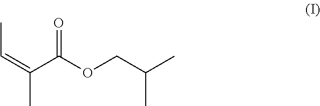

Isobutyl angelate is a compound obtained by esterifying (isobutyl-esterifying) angelic acid ((Z)-2-methyl-2-butenoic acid) represented by formula (II).

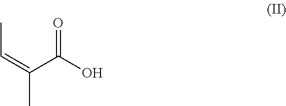

Isobutyl angelate may be a chemically synthesized product or a product derived from a natural material. Isobutyl angelate derived from a natural material is preferably one derived from Roman chamomile (Anthemis nobilis). Roman chamomile is a perennial plant belonging to the family Asteraceae. In an essential oil or an extract of Roman chamomile, isobutyl angelate, butyl angelate, isopropyl angelate and the like are contained as the main components.

An essential oil of Roman chamomile can be obtained by subjecting Roman chamomile to steam distillation, squeezing or the like. The part of Roman chamomile to be used is not limited. All parts including petals, leaves and stems can be used, and petals are preferably used.

An extract of Roman chamomile can be produced by subjecting Roman chamomile to reflux extraction, normal temperature homogenization extraction, supercritical fluid extraction or the like. The plant body part of Roman chamomile to be used is not limited. As in the case with the essential oil, all parts including petals, leaves and stems can be used, and petals are preferably used. In the case where Roman chamomile is extracted with a solvent like a reflux extraction method, specific examples of the solvent include: an organic solvent such as alcohols (e.g., a lower alcohol such as methanol and ethanol, or a polyhydric alcohol such as ethylene glycol, propylene glycol, 1,3-butylene glycol and glycerin), a glycerin fatty acid ester, ketones having a relatively high polarity (e.g., acetone) and esters (e.g., ethyl acetate); and water.

Each of the essential oil and the extract of Roman chamomile contains an angelate such as isobutyl angelate, and therefore can be used without any modification as the isobutyl angelate. Alternatively, the essential oil or the extract of Roman chamomile may be purified to remove impurities therefrom, or isobutyl angelate may be isolated from the essential oil or the extract of Roman chamomile for practical use.

The bitter taste inhibitor according to one embodiment of the present disclosure may contain other components, as long as the advantageous effect of the present invention cannot be impaired. The form of the bitter taste inhibitor according to one embodiment of the present disclosure is not particularly limited. For example, the bitter taste inhibitor may be prepared in the form of a liquid preparation or a powdery preparation.

In the case where the bitter taste inhibitor is used in the form of a liquid preparation, isobutyl angelate may be diluted with a solvent, for example. Specific examples of the solvent to be used for the dilution include water, ethanol, glycerin, propylene glycol, triacetin, a medium-chain triglyceride, and an animal or vegetable oil or fat. Alternatively, the bitter taste inhibitor may be combined with an auxiliary component or other active ingredient to prepare a mix. For example, the bitter taste inhibitor may be used in combination with any one of known various natural fragrances, synthetic fragrances and the like.

The bitter taste inhibitor according to one embodiment of the present disclosure may be mixed with an excipient (e.g., dextrin, gum arabic, lactose) and the above-mentioned solvent, and then a resultant mixture may be spray-dried to prepare a powdery or granular preparation or may be lyophilized or heat-dried to prepare a solid preparation. The bitter taste inhibitor according to one embodiment of the present disclosure may be prepared into various forms depending on the intended uses thereof.

The bitter taste to be inhibited by the bitter taste inhibitor according to one embodiment of the present disclosure includes a bitter taste derived from a foodstuff or a bitter taste derived from a bitter taste ingredient. Examples of the foodstuff having a bitter taste include coffee, tea, cacao, citrus (e.g., orange, grapefruit, bitter orange, yuzu (Citrus junos)) and hop.

Specific examples of the bitter taste ingredient include: alkaloids such as caffeine and theobromine; polyphenols such as catechin, chlorogenic acid and cacao polyphenol; limonoids such as limonin and nomilin; humulone compounds such as humulone and isohumulone; a bitter taste glycoside such as naringin and swertiamarin; inorganic salts such as a magnesium salt and a calcium salt; and a bitter taste protein or amino acid. Among these components, specific examples of a bitter taste ingredient other than the components that serve as bitter taste sources for the above-mentioned foodstuffs include L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian.

An amino acid other than glycine, such as L-leucine and L-isoleucine, has a stereoisomer (an L-form and a D-form). The taste of the L-form and the teste of the D-form are different from each other. For example, L-leucine has bitter taste, while D-leucine has a sweet taste. The substance of which the bitter taste is to be inhibited by the bitter taste inhibitor according to one embodiment is an L-form isomer having a bitter taste. When an L-form is contained, a mixture of an L-form and a D-form, such as a racemic form, can have a bitter taste derived from the L-form. On the other hand, Coptis rhizome, Phellodendron bark, Swertia herb, Atractylodes lancea rhizoma, Rhubarb and Gentian are crude drugs each having a bitter taste.

The bitter taste inhibitor according to one embodiment of the present disclosure can be used by being added to a food or beverage having a bitter taste. The amount of the bitter taste inhibitor to be added is adjusted appropriately depending on the type or amount of a foodstuff or a bitter taste ingredient contained in a food or beverage. For example, the bitter taste inhibitor is added to a food or beverage in an amount of at least 0.05 ppm by mass in terms of an isobutyl angelate content. From the viewpoint of the influence of a food or beverage itself, the amount of the bitter taste inhibitor to be added is about up to 10 ppm in terms of an isobutyl angelate content.

More specifically, the bitter taste inhibitor is generally added to a food or beverage having a bitter taste derived from the above-mentioned foodstuff in an amount of about 0.05 to 2 ppm by mass, preferably about 0.1 to 1 ppm by mass in terms of isobutyl angelate content. On the other hand, the bitter taste inhibitor is generally added to a food or beverage having a bitter taste derived from the above-mentioned bitter taste ingredient in an amount of about 0.05 to 4 ppm by mass, preferably about 0.1 to 2.5 ppm by mass, in terms of isobutyl angelate content.

The food or beverage having a bitter taste is not particularly limited, and specific examples of the food or beverage include coffee, a coffee beverage, coffee-containing sweet stuffs, oolong tea, a oolong tea beverage, green tea, a green tea beverage, green-tea- or powdered-green-tea-containing sweet stuffs, chocolate (high-cacao chocolate), chocolate sweet stuffs, a citrus juice (e.g., orange juice, grapefruit juice), marmalade, citrus-containing sweet stuffs, a citrus-containing seasoning agent (e.g., ponzu sauce), beer, a beer-taste beverage, a nutrition-supplement drink (e.g., a crude-drug-containing nutrition-supplement drink), a nutritional food, a functional food, a health food, an oriental medicine, and a gastrointestinal medicine. Particularly an orange fruit suffering from HLB disease (citrus greening disease) has an intensely bitter taste. Therefore, the bitter taste inhibitor according to one embodiment of the present disclosure can be used suitably for a food or beverage, e.g., a juice, prepared using an orange fruit juice.

When the bitter taste inhibitor according to one embodiment of the present disclosure is added to the above-mentioned food or beverage having a bitter taste, it becomes possible to produce a food or beverage having a reduced bitter taste without deteriorating the influence of the food or beverage itself. The bitter taste inhibitor according to one embodiment of the present disclosure can be used suitably in the fields of, for example, food industries, medical/quasi drug industries.

### EXAMPLES

Hereinbelow, the bitter taste inhibitor according to the present disclosure will be described specifically with reference to examples and comparative examples. However, the bitter taste inhibitor according to the present disclosure is not intended to be limited by these examples.

### Preparation Example 1

First, 0.4 g of isobutyl angelate was diluted with hydrous ethanol (ethanol concentration: 60% by mass) so that the total amount became 1000 g. The concentration of isobutyl angelate in the resultant solution was 400 ppm by mass.

### Preparation Example 2

First, 1.25 g of Roman chamomile essential oil was diluted with hydrous ethanol (ethanol concentration: 60% by mass) so that the total amount became 1000 g. Angelates contained in Roman chamomile essential oil used are mentioned below. In Roman chamomile essential oil, 32.9% by mass of the angelates were contained. The concentration of the angelates in the resultant solution was about 411 ppm by mass (isobutyl angelate: about 356 ppm by mass).

Propyl angelate: 2.9% by mass

Isobutyl angelate: 28.5% by mass

2-Methyl-2-butyl angelate: 1.5% by mass

Butyl angelate: a trace amount

tert-Butyl angelate: a trace amount

### Example 1: Inhibition of Bitter Taste in Beer

The resultant solution in Preparation Example 1 was added in an amount of 0.15% by mass to a beer for domestic and international sales. The concentration of isobutyl angelate added to the beer was 0.6 ppm by mass. The bitter taste of the beer having isobutyl angelate added thereto was evaluated in the following manner. The result is shown in Table 1.

The beer (without addition of isobutyl angelate) was tasted by seven panelists (one twenty-something male, one thirty-something male, one forty-something male, one fifty-something male, one sixty-something male, and one twenty-something female and one thirty-something female). Subsequently, the seven panelists rinsed their mouths sufficiently, and then the beer having isobutyl angelate added thereto (with addition of isobutyl angelate) was tasted by the seven panelists. The panelists compared the isobutyl-angelate-free beer with the isobutyl-angelate-containing beer, and then determined whether or not the bitter taste in the isobutyl-angelate-containing beer was improved compared with the isobutyl-angelate-free beer in accordance with the following criteria. When the sum total of the scores determined by the seven panelists was 10 points or greater, it was determined that the bitter taste was improved.

2 points: a bitter taste was not sensed, or a bitter taste was remarkably improved;

1 point: although a bitter taste was sensed, the bitter taste was improved compared with the isobutyl-angelate-free beer; and

0 point: the bitter taste was not improved.

### Example 2: Inhibition of Bitter Taste in Beer

The resultant solution in Preparation Example 2 was added in an amount of 0.15% by mass to a beer for domestic and international sales. The concentration of the angelates added to the beer was about 0.62 ppm by mass. Comparison was made between the beer (without addition of the angelates) and the beer having the angelates added thereto (with addition of the angelates), and the evaluation was carried out in the same manner as in Example 1. The result is shown in Table 1.

### Example 3: Inhibition of Bitter Taste in Grapefruit Juice

The resultant solution in Preparation Example 1 was added in an amount of 0.1% by mass to a 100% grapefruit juice. The concentration of isobutyl angelate added to the grapefruit juice was 0.4 ppm by mass. Comparison was made between the grapefruit juice (without addition of isobutyl angelate) and the grapefruit juice having isobutyl angelate added thereto (with addition of isobutyl angelate), and the evaluation was carried out in the same manner as in Example 1. The result is shown in Table 1.

### Example 4: Inhibition of Bitter Taste in Grapefruit Juice

The resultant solution in Preparation Example 2 was added in an amount of 0.1% by mass to a 100% grapefruit juice. The concentration of the angelates added to the grapefruit juice was about 0.41 ppm by mass. Comparison was made between the grapefruit juice (without addition of the angelates) and the grapefruit juice having angelates added thereto (with addition of angelates), and the evaluation was carried out in the same manner as in Example 1. The result is shown in Table 1.

### Example 5: Inhibition of Bitter Taste in Coffee

The resultant solution in Preparation Example 1 was added in an amount of 0.1% by mass to sugar-free black coffee. The concentration of isobutyl angelate added to the sugar-free black coffee was 0.4 ppm by mass.

Comparison was made between the sugar-free black coffee (without addition of isobutyl angelate) and the sugar-free black coffee having isobutyl angelate added thereto (with addition of isobutyl angelate), and the evaluation was carried out in the same manner as in Example 1. The result is shown in Table 1.

### Example 6: Inhibition of Bitter Taste in Coffee

The resultant solution in Preparation Example 2 was added in an amount of 0.1% by mass to sugar-free black coffee. The concentration of the angelates added to the sugar-free black coffee was about 0.41 ppm by mass. Comparison was made between the sugar-free black coffee (without addition of the angelates) and the sugar-free black coffee having the angelates added thereto (with addition of the angelates), and the evaluation was carried out in the same manner as in Example 1. The result is shown in Table 1.

### Example 7: Inhibition of Bitter Taste in Green Tea Beverage

The resultant solution in Preparation Example 1 was added in an amount of 0.05% by mass to a green tea beverage (containing polyphenol components in an amount of 35 mg or more per 100 ml). The concentration of isobutyl angelate added to the green tea beverage was 0.2 ppm by mass. Comparison was made between the green tea beverage (without addition of isobutyl angelate) and the green tea beverage having isobutyl angelate added thereto (with addition of isobutyl angelate), and the evaluation was carried out in the same manner as in Example 1. The result is shown in Table 1.

### Example 8: Inhibition of Bitter Taste in Green Tea Beverage

The resultant solution in Preparation Example 2 was added in an amount of 0.05% by mass to a green tea beverage. The concentration of the angelates added to the green tea beverage was about 0.21 ppm by mass. Comparison was made between the green tea beverage (without addition of the angelates) and the green tea beverage having the angelates added thereto (with addition of the angelates), and the evaluation was carried out in the same manner as in Example 1. The result is shown in Table 1.

### Example 9: Inhibition of Bitter Taste in High-Cacao Chocolate

The resultant solution in Preparation Example 1 was added in an amount of 0.2% by mass to high-cacao chocolate (cacao content: 86%). The concentration of isobutyl angelate added to the high-cacao chocolate was 0.8 ppm by mass. Comparison was made between the high-cacao chocolate (without addition of isobutyl angelate) and the high-cacao chocolate having isobutyl angelate added thereto (with addition of isobutyl angelate), and the evaluation was carried out in the same manner as in Example 1. The result is shown in Table 1.

### Example 10: Inhibition of Bitter Taste in High-Cacao Chocolate

The resultant solution in Preparation Example 2 was added in an amount of 0.2% by mass to high-cacao chocolate. The concentration of the angelates added to the high-cacao chocolate was about 0.82 ppm by mass. Comparison was made between the high-cacao chocolate (without addition of the angelates) and the high-cacao chocolate having the angelates added thereto (with addition of the angelates), and the evaluation was carried out in the same manner as in Example 1. The result is shown in Table 1.

### Example 11: Inhibition of Bitter Taste in Orange Juice

The resultant solution in Preparation Example 1 was added in an amount of 0.1% by mass to a 100% orange juice. The concentration of isobutyl angelate added to the orange juice was 0.4 ppm by mass. Comparison was made between the orange juice (without addition of isobutyl angelate) and the orange juice having isobutyl angelate added thereto (with addition of isobutyl angelate), and the evaluation was carried out in the same manner as in Example 1. The result is shown in Table 1.

### Example 12: Inhibition of Bitter Taste in Orange Juice

The resultant solution in Preparation Example 2 was added in an amount of 0.1% by mass to a 100% orange juice. The concentration of the angelates added to the orange juice was about 0.41 ppm by mass. Comparison was made between the orange juice (without addition of the angelates) and the orange juice having the angelates added thereto (with addition of the angelates), and the evaluation was carried out in the same manner as in Example 1. The result is shown in Table 1.

### Example 13: Inhibition of Bitter Taste in Orange Juice

A 100% orange juice containing 5 ppm of limonin and 5 ppm of nomilin was prepared. The resultant solution in Preparation Example 1 was added in an amount of 0.2% by mass to the prepared orange juice. The concentration of isobutyl angelate added to the prepared orange juice was 0.8 ppm by mass. Comparison was made between the prepared orange juice (without addition of isobutyl angelate) and the prepared orange juice having the isobutyl angelate added thereto (with addition of the isobutyl angelate), and the evaluation was carried out in the same manner as in Example 1. The result is shown in Table 1.

### Example 14: Inhibition of Bitter Taste in Orange Juice

A 100% orange juice containing 5 ppm of limonin and 5 ppm of nomilin was prepared. The resultant solution in Preparation Example 2 was added in an amount of 0.2% by mass to the prepared orange juice. The concentration of the angelates added to the prepared orange juice was about 0.82 ppm by mass. Comparison was made between the prepared orange juice (without addition of angelates) and the prepared orange juice having the angelates added thereto (with addition of the angelates), and the evaluation was carried out in the same manner as in Example 1. The result is shown in Table 1.

TABLE 1

| | Food or beverage | Bitter taste inhibitor | | |
|---|---|---|---|---|
| | having bitter taste | (% by mass) | Evaluation | |
| Example | Beer | Preparation Example 1 (0.15) | 14 | A characteristic bitter |
| 1 | | (0.6 ppm by mass) | points | taste derived from hop |
| Example | | Preparation Example 2 (0.15) | 14 | was reduced. |
| 2 | | (about 0.62 ppm by mass) | points | |
| Example | 100% | Preparation Example 1 (0.1) | 14 | A characteristic bitter |
| 3 | Grapefruit juice | (0.4 ppm by mass) | points | taste derived from |
| Example | | Preparation Example 2 (0.1) | 14 | naringin was reduced. |
| 4 | | (about 0.41 ppm by mass) | points | |
| Example | Sugar-free | Preparation Example 1 (0.1) | 12 | A characteristic bitter |
| 5 | black coffee | (0.4 ppm by mass) | points | taste derived from |
| Example | | Preparation Example 2 (0.1) | 12 | caffeine and chlorogenic |
| 6 | | (about 0.41 ppm by mass) | points | acid was reduced. |
| Example | Green tea beverage | Preparation Example 1 (0.05) | 13 | A characteristic bitter |
| 7 | | (0.2 ppm by mass) | points | taste derived from |
| Example | | Preparation Example 2 (0.05) | 13 | catechin was reduced. |
| 8 | | (about 0.21 ppm by mass) | points | |
| Example | Chocolate having | Preparation Example 1 (0.2) | 12 | A characteristic bitter |
| 9 | cacao content of 86% | (0.8 ppm by mass) | points | taste derived from |
| Example | | Preparation Example 2 (0.2) | 12 | theobromine was reduced. |
| 10 | | (about 0.82 ppm by mass) | points | |
| Example | 100% | Preparation Example 1 (0.1) | 14 | Although the bitter taste |
| 11 | Orange juice | (0.4 ppm by mass) | points | was originally weak, the |
| Example | | Preparation Example 2 (0.1) | 14 | bitter taste was reduced. |
| 12 | | (about 0.41 ppm by mass) | points | |
| Example | 100% | Preparation Example 1 (0.2) | 11 | An intense and lasting |
| 13 | Orange juice^{(note 1)} | (0.8 ppm by mass) | points | bitter taste derived from |
| Example | | Preparation Example 2 (0.2) | 11 | limonin and nomilin was |
| 14 | | (about 0.82 ppm by mass) | points | greatly reduced. |

The bitter taste inhibitor concentration shown in each of the lower columns represents the concentration of isobutyl angelate or the angelates.
^{(note 1)}Each of limonin and nomilin was contained at a concentration of 5 ppm.

As shown in Table 1, it is demonstrated that, when isobutyl angelate or Roman chamomile essential oil containing angelates was added to a food or beverage having a bitter taste, the bitter taste is reduced without deteriorating the influence of the food or beverage itself.

### Preparation Example 3

First, 1 g of isobutyl angelate was diluted with hydrous ethanol (ethanol concentration: 60% by mass) to a total amount of 1000 g. The concentration of isobutyl angelate in the resultant solution was 1000 ppm by mass.

### Preparation Example 4

First, 3.05 g of Roman chamomile essential oil used in Preparation Example 2 was diluted with hydrous ethanol (ethanol concentration: 60% by mass) to a total amount of 1000 g. The concentration of isobutyl angelate in the resultant solution was about 869 ppm by mass.

### Example 15: Inhibition of Bitter Taste in Glycine

Glycine (manufactured by Kyowa Hakko Bio Co., Ltd.) was diluted with purified water to prepare a 1% by mass aqueous glycine solution. The resultant solution in Preparation Example 3 was added in an amount of 0.05% by mass to the aqueous solution. The concentration of isobutyl angelate added to the aqueous solution was 0.5 ppm by mass. Comparison was made between the aqueous glycine solution (without addition of isobutyl angelate) and the aqueous glycine solution having isobutyl angelate added thereto (with addition of isobutyl angelate), and the evaluation was carried out in the same manner as in Example 1. The result is shown in Table 2.

### Example 16: Inhibition of Bitter Taste in Glycine

The resultant solution in Preparation Example 4 was added in an amount of 0.05% by mass to a 1% by mass aqueous glycine solution prepared in the same manner as in Example 15. The concentration of isobutyl angelate added to the aqueous solution was about 0.43 ppm by mass. Comparison was made between the aqueous glycine solution (without addition of isobutyl angelate) and the aqueous glycine solution having isobutyl angelate added thereto (with addition of isobutyl angelate), and the evaluation was carried out in the same manner as in Example 1. The result is shown in Table 2.

### Example 17: Inhibition of Bitter Taste in L-Leucine

L-Leucine (manufactured by Kyowa Hakko Bio Co., Ltd.) was diluted with purified water to prepare a 1% by mass aqueous L-leucine solution. The resultant solution in Preparation Example 3 was added in an amount of 0.1% by mass to the aqueous solution. The concentration of isobutyl angelate added to the aqueous solution was 1 ppm by mass. Comparison was made between the aqueous L-leucine solution (without addition of isobutyl angelate) and the aqueous L-leucine solution having isobutyl angelate added thereto (with addition of isobutyl angelate), and the evaluation was carried out in the same manner as in Example 1. The result is shown in Table 2.

### Example 18: Inhibition of Bitter Taste in L-Leucine

The resultant solution in Preparation Example 4 was added in an amount of 0.1% by mass to a 1% by mass aqueous L-leucine solution prepared in the same manner as in Example 17. The concentration of isobutyl angelate added to the aqueous solution was about 0.87 ppm by mass. Comparison was made between the aqueous L-leucine solution (without addition of isobutyl angelate) and the aqueous L-leucine solution having isobutyl angelate added thereto (with addition of isobutyl angelate), and the evaluation was carried out in the same manner as in Example 1. The result is shown in Table 2.

### Example 19: Inhibition of Bitter Taste in L-Phenylalanine

L-Phenylalanine (manufactured by Kyowa Hakko Bio Co., Ltd.) was diluted with purified water to prepare a 1% by mass aqueous L-phenylalanine solution. The resultant solution in Preparation Example 3 was added in an amount of 0.2% by mass to the aqueous solution. The concentration of isobutyl angelate added to the aqueous solution was 2 ppm by mass. Comparison was made between the aqueous L-phenylalanine solution (without addition of isobutyl angelate) and the aqueous L-phenylalanine solution having isobutyl angelate added thereto (with addition of isobutyl angelate), and the evaluation was carried out in the same manner as in Example 1. The result is shown in Table 2.

### Example 20: Inhibition of Bitter Taste in L-Phenylalanine

The resultant solution in Preparation Example 4 was added in an amount of 0.2% by mass to a 1% by mass aqueous L-phenylalanine solution prepared in the same manner as in Example 19. The concentration of isobutyl angelate added to the aqueous solution was about 1.74 ppm by mass. Comparison was made between the aqueous L-phenylalanine solution (without addition of isobutyl angelate) and the aqueous L-phenylalanine solution having isobutyl angelate added thereto (with addition of isobutyl angelate), and the evaluation was carried out in the same manner as in Example 1. The result is shown in Table 2.

### Example 21: Inhibition of Bitter Taste in L-Alanine

L-Alanine (manufactured by Kyowa Hakko Bio Co., Ltd.) was diluted with purified water to prepare a 1% by mass aqueous L-alanine solution. The resultant solution in Preparation Example 3 was added in an amount of 0.05% by mass to the aqueous solution. The concentration of isobutyl angelate added to the aqueous solution was 0.5 ppm by mass. Comparison was made between the aqueous L-alanine solution (without addition of isobutyl angelate) and the aqueous L-alanine solution having isobutyl angelate added thereto (with addition of isobutyl angelate), and the evaluation was carried out in the same manner as in Example 1. The result is shown in Table 2.

### Example 22: Inhibition of Bitter Taste in L-Alanine

The resultant solution in Preparation Example 4 was added in an amount of 0.05% by mass to a 1% by mass aqueous L-alanine solution prepared in the same manner as in Example 21. The concentration of isobutyl angelate added to the aqueous solution was about 0.43 ppm by mass. Comparison was made between the aqueous L-alanine solution (without addition of isobutyl angelate) and the aqueous L-alanine solution having isobutyl angelate added thereto (with addition of isobutyl angelate), and the evaluation was carried out in the same manner as in Example 1. The result is shown in Table 2.

### Example 23: Inhibition of Bitter Taste in L-Isoleucine

L-Isoleucine (manufactured by Kyowa Hakko Bio Co., Ltd.) was diluted with purified water to prepare a 1% by mass aqueous L-isoleucine solution. The resultant solution in Preparation Example 3 was added in an amount of 0.07% by mass to the aqueous solution. The concentration of isobutyl angelate added to the aqueous solution was 0.7 ppm by mass. Comparison was made between the aqueous L-isoleucine solution (without addition of isobutyl angelate) and the aqueous L-isoleucine solution having isobutyl angelate added thereto (with addition of isobutyl angelate), and the evaluation was carried out in the same manner as in Example 1. The result is shown in Table 2.

### Example 24: Inhibition of Bitter Taste in L-Isoleucine

The resultant solution in Preparation Example 4 was added in an amount of 0.07% by mass to a 1% by mass aqueous L-isoleucine solution prepared in the same manner as in Example 23. The concentration of isobutyl angelate added to the aqueous solution was about 0.61 ppm by mass. Comparison was made between the aqueous L-isoleucine solution (without addition of isobutyl angelate) and the aqueous L-isoleucine solution having isobutyl angelate added thereto (with addition of isobutyl angelate), and the evaluation was carried out in the same manner as in Example 1. The result is shown in Table 2.

### Example 25: Inhibition of Bitter Taste in L-Valine

L-Valine (manufactured by Kyowa Hakko Bio Co., Ltd.) was diluted with purified water to prepare a 1% by mass aqueous L-valine solution. The resultant solution in Preparation Example 3 was added in an amount of 0.05% by mass to the aqueous solution. The concentration of isobutyl angelate added to the aqueous solution was 0.5 ppm by mass. Comparison was made between the aqueous L-valine solution (without addition of isobutyl angelate) and the aqueous L-valine solution having isobutyl angelate added thereto (with addition of isobutyl angelate), and the evaluation was carried out in the same manner as in Example 1. The result is shown in Table 2.

### Example 26: Inhibition of Bitter Taste in L-Valine

The resultant solution in Preparation Example 4 was added in an amount of 0.05% by mass to a 1% by mass aqueous L-valine solution prepared in the same manner as in Example 25. The concentration of isobutyl angelate added to the aqueous solution was about 0.43 ppm by mass. Comparison was made between the aqueous L-valine solution (without addition of isobutyl angelate) and the aqueous L-valine solution having isobutyl angelate added thereto (with addition of isobutyl angelate), and the evaluation was carried out in the same manner as in Example 1. The result is shown in Table 2.

### Example 27: Inhibition of Bitter Taste in L-Proline

L-Proline (manufactured by Kyowa Hakko Bio Co., Ltd.) was diluted with purified water to prepare a 1% by mass aqueous L-proline solution. The resultant solution in Preparation Example 3 was added in an amount of 0.05% by mass to the aqueous solution. The concentration of isobutyl angelate added to the aqueous solution was 0.5 ppm by mass. Comparison was made between the aqueous L-proline solution (without addition of isobutyl angelate) and the aqueous L-proline solution having isobutyl angelate added thereto (with addition of isobutyl angelate), and the evaluation was carried out in the same manner as in Example 1. The result is shown in Table 2.

### Example 28: Inhibition of Bitter Taste in L-Proline

The resultant solution in Preparation Example 4 was added in an amount of 0.05% by mass to a 1% by mass aqueous L-proline solution prepared in the same manner as in Example 27. The concentration of isobutyl angelate added to the aqueous solution was about 0.43 ppm by mass. Comparison was made between the aqueous L-proline solution (without addition of isobutyl angelate) and the aqueous L-proline solution having isobutyl angelate added thereto (with addition of isobutyl angelate), and the evaluation was carried out in the same manner as in Example 1. The result is shown in Table 2.

### Example 29: Inhibition of Bitter Taste in L-Tryptophan

L-Tryptophan (manufactured by Kyowa Hakko Bio Co., Ltd.) was diluted with purified water to prepare a 1% by mass aqueous L-tryptophan solution. The resultant solution in Preparation Example 3 was added in an amount of 0.05% by mass to the aqueous solution. The concentration of isobutyl angelate added to the aqueous solution was 0.5 ppm by mass. Comparison was made between the aqueous L-tryptophan solution (without addition of isobutyl angelate) and the aqueous L-tryptophan solution having isobutyl angelate added thereto (with addition of isobutyl angelate), and the evaluation was carried out in the same manner as in Example 1. The result is shown in Table 2.

### Example 30: Inhibition of Bitter Taste in L-Tryptophan

The resultant solution in Preparation Example 4 was added in an amount of 0.05% by mass to a 1% by mass aqueous L-tryptophan solution prepared in the same manner as in Example 29. The concentration of isobutyl angelate added to the aqueous solution was about 0.43 ppm by mass. Comparison was made between the aqueous L-tryptophan solution (without addition of isobutyl angelate) and the aqueous L-tryptophan solution having isobutyl angelate added thereto (with addition of isobutyl angelate), and the evaluation was carried out in the same manner as in Example 1. The result is shown in Table 2.

TABLE 2

| | Aqueous amino acid | Bitter taste inhibitor | | |
|---|---|---|---|---|
| | solution | (% by mass) | Evaluation | |
| Example | 1% By mass aqueous | Preparation Example 3 (0.05) | 14 | Very little bitter taste |
| 15 | glycine solution | (0.5 ppm by mass) | points | was sensed. |
| Example | | Preparation Example 4 (0.05) | 14 | |
| 16 | | (about 0.43 ppm by mass) | points | |
| Example | 1% By mass aqueous | Preparation Example 3 (0.1) | 14 | Very little bitter taste |
| 17 | L-leucine solution | (1 ppm by mass) | points | was sensed. |
| Example | | Preparation Example 4 (0.1) | 14 | |
| 18 | | (about 0.87 ppm by mass) | points | |
| Example | 1% By mass aqueous | Preparation Example 3 (0.2) | 13 | Although a slight bitter |
| 19 | L-phenylalanine | (2 ppm by mass) | points | taste was sensed, the |
| Example | solution | Preparation Example 4 (0.2) | 12 | bitter taste was not |
| 20 | | (about 1.74 ppm by mass) | points | particularly noticeable |
| | | | | and was clearly reduced. |
| Example | 1% By mass aqueous | Preparation Example 3 (0.05) | 14 | Very little bitter taste |
| 21 | L-alanine solution | (0.5 ppm by mass) | points | was sensed, and a slight |
| Example | | Preparation Example 4 (0.05) | 14 | sweet taste derived from |
| 22 | | (about 0.43 ppm by mass) | points | alanine was sensed. |
| Example | 1% By mass aqueous | Preparation Example 3 (0.07) | 13 | Very little bitter taste |
| 23 | L-isoleucine | (0.7 ppm by mass) | points | was sensed. |
| Example | solution | Preparation Example 4 (0.07) | 13 | |
| 24 | | (about 0.61 ppm by mass) | points | |
| Example | 1% By mass aqueous | Preparation Example 3 (0.05) | 14 | Very little bitter taste |
| 25 | L-valine solution | (0.5 ppm by mass) | points | was sensed. |
| Example | | Preparation Example 4 (0.05) | 14 | |
| 26 | | (about 0.43 ppm by mass) | points | |
| Example | 1% By mass aqueous | Preparation Example 3 (0.05) | 14 | Very little bitter taste |
| 27 | L-proline solution | (0.5 ppm by mass) | points | was sensed, and an impure |
| Example | | Preparation Example 4 (0.05) | 14 | taste derived from |
| 28 | | (about 0.43 ppm by mass) | points | proline was clearly |
| | | | | reduced. |
| Example | 1% By mass aqueous | Preparation Example 3 (0.05) | 12 | Although a slight bitter |
| 29 | L-tryptophan | (0.5 ppm by mass) | points | taste was sensed, the |
| Example | solution | Preparation Example 4 (0.05) | 12 | bitter taste was not |
| 30 | | (about 0.43 ppm by mass) | points | particularly noticeable |
| | | | | and was clearly reduced. |

As shown in Table 2, it is demonstrated that, when isobutyl angelate or Roman chamomile essential oil containing isobutyl angelate is added to an amino acid having a bitter taste, the bitter taste derived from the amino acid is reduced.

### Example 31: Inhibition of Bitter Taste in Coptis rhizome┌Coptis rhizome:┘ (a t h e

Coptis rhizome powder according to the Japanese Pharmacopoeia (manufactured by Nippon Funmatsu Yakuhin Co., Ltd.) was added in an amount of 0.1 g to 100 mL of hot water (85° C.), and the resultant solution was subjected to an extraction process for 30 minutes. Subsequently, a supernatant was recovered and cooled to room temperature to obtain an extract of Coptis rhizome powder. The resultant solution in Preparation Example 3 was added in an amount of 0.2% by mass to the extract of Coptis rhizome powder. The concentration of isobutyl angelate added to the aqueous solution was 2 ppm by mass. Comparison was made between the extract of Coptis rhizome powder (without addition of isobutyl angelate) and the extract of Coptis rhizome powder having isobutyl angelate added thereto (with addition of isobutyl angelate), and the evaluation was carried out in the same manner as in Example 1. The result is shown in Table 3.

### Example 32: Inhibition of Bitter Taste in Coptis rhizome

The resultant solution in Preparation Example 4 was added in an amount of 0.2% by mass to an extract of Coptis rhizome powder prepared in the same manner as in Example 31. The concentration of isobutyl angelate added to the aqueous solution was about 1.74 ppm by mass. Comparison was made between the extract of Coptis rhizome powder (without addition of isobutyl angelate) and the extract of Coptis rhizome powder having isobutyl angelate added thereto (with addition of isobutyl angelate), and the evaluation was carried out in the same manner as in Example 1. The result is shown in Table 3.

### Example 33: Inhibition of Bitter Taste in Phellodendron Bark

Phellodendron bark powder according to the Japanese Pharmacopoeia (manufactured by Nippon Funmatsu Yakuhin Co., Ltd.) was added in an amount of 0.1 g to 100 mL of hot water (85° C.), and the resultant solution was subjected to an extraction process for 30 minutes. Subsequently, a supernatant was recovered and cooled to room temperature to obtain an extract of Phellodendron bark powder. The resultant solution in Preparation Example 3 was added in an amount of 0.2% by mass to the extract of Phellodendron bark powder. The concentration of isobutyl angelate added to the aqueous solution was 2 ppm by mass. Comparison was made between the extract of Phellodendron bark powder (without addition of isobutyl angelate) and the extract of Phellodendron bark powder having isobutyl angelate added thereto (with addition of isobutyl angelate), and the evaluation was carried out in the same manner as in Example 1. The result is shown in Table 3.

### Example 34: Inhibition of Bitter Taste in Phellodendron Bark

The resultant solution in Preparation Example 4 was added in an amount of 0.2% by mass to an extract of Phellodendron bark powder prepared in the same manner as in Example 33. The concentration of isobutyl angelate added to the aqueous solution was about 1.74 ppm by mass. Comparison was made between the extract of Phellodendron bark powder (without addition of isobutyl angelate) and the extract of Phellodendron bark powder having isobutyl angelate added thereto (with addition of isobutyl angelate), and the evaluation was carried out in the same manner as in Example 1. The result is shown in Table 3.

### Example 35: Inhibition of Bitter Taste in Swertia Herb

Swertia herb powder according to the Japanese Pharmacopoeia (manufactured by Nippon Funmatsu Yakuhin Co., Ltd.) was added in an amount of 0.1 g to 100 mL of hot water (85° C.), and the resultant solution was subjected to an extraction process for 30 minutes. Subsequently, a supernatant was recovered and cooled to room temperature to obtain an extract of Swertia herb powder. The resultant solution in Preparation Example 3 was added in an amount of 0.2% by mass to the extract of Swertia herb powder. The concentration of isobutyl angelate added to the aqueous solution was 2 ppm by mass. Comparison was made between the extract of Swertia herb powder (without addition of isobutyl angelate) and the extract of Swertia herb powder having isobutyl angelate added thereto (with addition of isobutyl angelate), and the evaluation was carried out in the same manner as in Example 1. The result is shown in Table 3.

### Example 36: Inhibition of Bitter Taste in Swertia Herb

The resultant solution in Preparation Example 4 was added in an amount of 0.2% by mass to an extract of Swertia herb powder prepared in the same manner as in Example 35. The concentration of isobutyl angelate added to the aqueous solution was about 1.74 ppm by mass. Comparison was made between the extract of Swertia herb powder (without addition of isobutyl angelate) and the extract of Swertia herb powder having isobutyl angelate added thereto (with addition of isobutyl angelate), and the evaluation was carried out in the same manner as in Example 1. The result is shown in Table 3.

### Example 37: Inhibition of Bitter Taste in Gentian

Gentian powder according to the Japanese Pharmacopoeia (manufactured by Nippon Funmatsu Yakuhin Co., Ltd.) was added in an amount of 0.1 g to 100 mL of hot water (85° C.), and the resultant solution was subjected to an extraction process for 30 minutes. Subsequently, a supernatant was recovered and cooled to room temperature to obtain an extract of Gentian powder. The resultant solution in Preparation Example 3 was added in an amount of 0.2% by mass to the extract of Gentian powder. The concentration of isobutyl angelate added to the aqueous solution was 2 ppm by mass. Comparison was made between the extract of Gentian powder (without addition of isobutyl angelate) and the extract of Gentian powder having isobutyl angelate added thereto (with addition of isobutyl angelate), and the evaluation was carried out in the same manner as in Example 1. The result is shown in Table 3.

### Example 38: Inhibition of Bitter Taste in Gentian

The resultant solution in Preparation Example 4 was added in an amount of 0.2% by mass to an extract of Gentian powder prepared in the same manner as in Example 37. The concentration of isobutyl angelate added to the aqueous solution was about 1.74 ppm by mass. Comparison was made between the extract of Gentian powder (without addition of isobutyl angelate) and the extract of Gentian powder having isobutyl angelate added thereto (with addition of isobutyl angelate), and the evaluation was carried out in the same manner as in Example 1. The result is shown in Table 3.

### Example 39: Inhibition of Bitter Taste in Atractylodes Lancea Rhizoma

Atractylodes lancea rhizoma powder according to the Japanese Pharmacopoeia (manufactured by Nippon Funmatsu Yakuhin Co., Ltd.) was added in an amount of 1 g to 100 mL of hot water (85° C.), and the resultant solution was subjected to an extraction process for 30 minutes. Subsequently, a supernatant was recovered and cooled to room temperature to obtain an extract of Atractylodes lancea rhizoma powder. The resultant solution in Preparation Example 3 was added in an amount of 0.1% by mass to the extract of Atractylodes lancea rhizoma powder. The concentration of isobutyl angelate added to the aqueous solution was 1 ppm by mass. Comparison was made between the extract of Atractylodes lancea rhizoma powder (without addition of isobutyl angelate) and the extract of Atractylodes lancea rhizoma powder having isobutyl angelate added thereto (with addition of isobutyl angelate), and the evaluation was carried out in the same manner as in Example 1. The result is shown in Table 3.

### Example 40: Inhibition of Bitter Taste in Atractylodes Lancea Rhizoma

The resultant solution in Preparation Example 4 was added in an amount of 0.1% by mass to an extract of Atractylodes lancea rhizoma powder prepared in the same manner as in Example 39. The concentration of isobutyl angelate added to the aqueous solution was about 0.87 ppm by mass. Comparison was made between the extract of Atractylodes lancea rhizoma powder (without addition of isobutyl angelate) and the extract of Atractylodes lancea rhizoma powder having isobutyl angelate added thereto (with addition of isobutyl angelate), and the evaluation was carried out in the same manner as in Example 1. The result is shown in Table 3.

### Example 41: Inhibition of Bitter Taste in Rhubarb

Rhubarb powder according to the Japanese Pharmacopoeia (manufactured by Nippon Funmatsu Yakuhin Co., Ltd.) was added in an amount of 1 g to 100 mL of hot water (85° C.), and the resultant solution was subjected to an extraction process for 30 minutes. Subsequently, a supernatant was recovered and cooled to room temperature to obtain an extract of Rhubarb powder. The resultant solution in Preparation Example 3 was added in an amount of 0.1% by mass to the extract of Rhubarb powder. The concentration of isobutyl angelate added to the aqueous solution was 1 ppm by mass. Comparison was made between the extract of Rhubarb powder (without addition of isobutyl angelate) and the extract of Rhubarb powder having isobutyl angelate added thereto (with addition of isobutyl angelate), and the evaluation was carried out in the same manner as in Example 1. The result is shown in Table 3.

### Example 42: Inhibition of Bitter Taste in Rhubarb

The resultant solution in Preparation Example 4 was added in an amount of 0.1% by mass to an extract of Rhubarb powder prepared in the same manner as in Example 41. The concentration of isobutyl angelate added to the aqueous solution was about 0.87 ppm by mass. Comparison was made between the extract of Rhubarb powder (without addition of isobutyl angelate) and the extract of Rhubarb powder having isobutyl angelate added thereto (with addition of isobutyl angelate), and the evaluation was carried out in the same manner as in Example 1. The result is shown in Table 3.

TABLE 3

| | | Bitter taste inhibitor | | |
|---|---|---|---|---|
| | Crude drug | (% by mass) | Evaluation | |
| Example | Coptis rhizome | Preparation Example 3 (0.2) | 12 | Although a slight bitter |
| 31 | extract | (2 ppm by mass) | points | taste was sensed, the |
| Example | | Preparation Example 4 (0.2) | 12 | bitter taste was not |
| 32 | | (about 1.74 ppm by mass) | points | particularly noticeable |
| | | | | and was clearly reduced. |
| Example | Phellodendron | Preparation Example 3 (0.2) | 13 | Very little bitter taste |
| 33 | bark extract | (2 ppm by mass) | points | was sensed. |
| Example | | Preparation Example 4 (0.2) | 12 | |
| 34 | | (about 1.74 ppm by mass) | points | |
| Example | Swertia herb | Preparation Example 3 (0.2) | 13 | Very little bitter taste |
| 35 | extract | (2 ppm by mass) | points | was sensed. |
| Example | | Preparation Example 4 (0.2) | 12 | |
| 36 | | (about 1.74 ppm by mass) | points | |
| Example | Gentian | Preparation Example 3 (0.2) | 13 | Very little bitter taste |
| 37 | extract | (2 ppm by mass) | points | was sensed. |
| Example | | Preparation Example 4 (0.2) | 13 | |
| 38 | | (about 1.74 ppm by mass) | points | |
| Example | Atractylodes | Preparation Example 3 (0.1) | 14 | Very little bitter taste |
| 39 | lancea rhizoma | (1 ppm by mass) | points | was sensed. |
| Example | extract | Preparation Example 4 (0.1) | 14 | |
| 40 | | (about 0.87 ppm by mass) | points | |
| Example | Rhubarb | Preparation Example 3 (0.1) | 14 | Although a slight sour |
| 41 | extract | (1 ppm by mass) | points | taste and a slight |
| Example | | Preparation Example 4 (0.1) | 14 | astringent taste were |
| 42 | | (about 0.87 ppm by mass) | points | sensed, very little |
| | | | | bitter taste was sensed. |

As shown in Table 3, it is demonstrated that, when isobutyl angelate or Roman chamomile essential oil containing isobutyl angelate is added to an extract of a crude drug having a bitter taste, a bitter taste derived from the crude drug is reduced.

What is claimed is:

1. A bitter taste inhibitor comprising:
isobutyl angelate as an active ingredient, and
at least one solvent or excipient selected from the group consisting of ethanol, hydrous ethanol, glycerin, hydrous glycerin, propylene glycol, hydrous propylene glycol, vegetable oil, medium-chain triglyceride, triacetin dextrin and gum arabic,
which is effective to inhibit a bitter taste derived from at least one food stuff selected from the group consisting of citrus, coffee, tea, cacao and hop, or
which is effective to inhibit a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian.

2. The bitter taste inhibitor according to claim 1, wherein the isobutyl angelate is an ester derived from Roman chamomile (Anthemis nobilis).

3. A food or beverage having a reduced bitter taste, comprising:
a food or beverage having a bitter taste derived from at least one foodstuff selected from the group consisting of citrus, coffee, tea, cacao and hop; and
the bitter taste inhibitor according to claim 1,
wherein the bitter taste inhibitor is contained in an amount of 0.05 to 2 ppm by mass in terms of an isobutyl angelate content.

4. A method for inhibiting a bitter taste derived from at least one foodstuff selected from the group consisting of citrus, coffee, tea, cacao and hop in a food or beverage having the bitter taste, the method comprising adding 0.05 to 2 ppm by mass of isobutyl angelate to the food or beverage.

5. The method according to claim 4, wherein the isobutyl angelate is an ester derived from Roman chamomile (Anthemis nobilis).

6. A bitter taste inhibitor comprising isobutyl angelate as an active ingredient and a solvent or an excipient according to claim 1, which is effective to inhibit a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian.

7. The bitter taste inhibitor according to claim 6, wherein the isobutyl angelate is an ester derived from Roman chamomile (Anthemis nobilis).

8. A food or beverage having a reduced bitter taste, comprising:
a food or beverage having a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian; and
the bitter taste inhibitor according to claim 6,
wherein the bitter taste inhibitor is contained in an amount of 0.05 to 4 ppm by mass in terms of an isobutyl angelate content.

9. A method for inhibiting a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian in a food or beverage having the bitter taste, the method comprising adding 0.05 to 4 ppm by mass of isobutyl angelate to the food or beverage.

10. The method according to claim 9, wherein the isobutyl angelate is an ester derived from Roman chamomile (Anthemis nobilis).

11. A bitter taste inhibitor comprising isobutyl angelate as an active ingredient and a solvent or an excipient according to claim 1, which is effective to inhibit a bitter taste derived from at least one food stuff selected from the group consisting of citrus, coffee, tea, cacao and hop.

12. The bitter taste inhibitor according to claim 11, wherein the isobutyl angelate is an ester derived from Roman chamomile (Anthemis nobilis).

13. A method for inhibiting a bitter taste derived from at least one foodstuff selected from the group consisting of citrus, coffee, tea, cacao and hop in a food or beverage having the bitter taste, the method comprising adding 0.05 to 2 ppm by mass of the bitter taste inhibitor according to claim 1 to the food or beverage.

14. A method for inhibiting a bitter taste derived from at least one bitter taste ingredient selected from the group consisting of L-leucine, L-isoleucine, L-valine, L-tryptophan, L-phenylalanine, L-alanine, glycine, L-proline, Coptis rhizome, Phellodendron bark, Swertia herb, Atractylodes lancea rhizoma, Rhubarb and Gentian in a food or beverage having the bitter taste, the method comprising adding 0.05 to 4 ppm by mass of the bitter taste inhibitor according to claim 1 to the food or beverage.
